SEQUENCE LISTING (4 sequences)

SEQ ID NO: 1 - DNA, 20 bp, Artificial Sequence
  Synthetic oligonucleotide primer for MMP-1
tgtggtgtctcacagcttcc

SEQ ID NO: 2 - DNA, 20 bp, Artificial Sequence
  Synthetic oligonucleotide primer for MMP-1
cacatcaggcactccacatc

SEQ ID NO: 3 - DNA, 24 bp, Artificial Sequence
  Synthetic oligonucleotide primer for GAPDH
tcggagtcaacggatttggtcgta

SEQ ID NO: 4 - DNA, 21 bp, Artificial Sequence
  Synthetic oligonucleotide primer for GAPDH
atggactgtggtcatgagtcc

(19) United States
(12) Patent Application Publication
(10) Pub. No.: US 2009/0142330 A1
(43) Pub. Date: Jun. 4, 2009

(54) Use of inactive-plasmin to treat chronic inflammatory disease and tumors

(75) Inventors: Yahong Zhang, North Potomac, MD (US); Larry M. Wahl, North Potomac, MD (US)

Correspondence Address: KLARQUIST SPARKMAN, LLP, 121 S.W. SALMON STREET, SUITE #1600, PORTLAND, OR, 97204-2988, US

(73) Assignees: The Government of the United States of America as represented by the Secretary of the Department of; Health and Human Services

(21) Appl. No.: 12/287,912

(22) Filed: Oct. 14, 2008

Related U.S. Application Data
(60) Provisional application No. 60/980,009, filed on Oct. 15, 2007.

(51) Int. Cl. A61K 38/48; A61P 29/00; A61P 35/00

(52) U.S. Cl. 424/94.64

(57) ABSTRACT

Methods are provided for the suppression of inflammation or a tumor. The methods can include selecting a subject in need of suppression of inflammation or the tumor and inhibiting plasmin activity in the subject to decrease matrix metalloproteinase production, thereby suppressing the inflammation or tumor. In several examples, an agent including inactive plasmin at a therapeutically effective concentration is administered to inhibit plasmin activity. Methods are also provided for modulating annexin A2 receptor activity.

### CROSS REFERENCE TO RELATED APPLICATION

This application claims the benefit of priority under 35 U.S.C. §119 to U.S. Provisional Application No. 60/980,009 filed on Oct. 15, 2007, which is incorporated herein by reference in its entirety.

### FIELD OF THE DISCLOSURE

This disclosure relates to the fields of inflammatory disease and tumors, specifically to the use of inactive-plasmin for suppressing an inflammatory disease or a tumor, such as cancer.

### BACKGROUND

Excessive breakdown of connective tissue is a feature of many pathological conditions. Such pathological conditions can include tumors (such as cancer) and inflammatory diseases. For example, excessive disintegration of connective tissue is a mechanism by which tumor cells invade and spread to other organs. Specific examples of inflammatory diseases associated with extensive connective tissue breakdown include atherosclerosis, periodontitis, and rheumatoid arthritis.

Atherosclerosis is a chronic inflammatory disease affecting arterial blood vessels. This disease most commonly becomes symptomatic when interfering with the coronary circulation supplying the heart or cerebral circulation supplying the brain. Atherosclerosis is the most important underlying cause of strokes, heart attacks, various heart diseases including congestive heart failure and most cardiovascular diseases in general.

Periodontitis is an inflammatory disease affecting the tissues that surround and support the teeth. This disease involves progressive loss of bone around teeth, which may lead to loosening and eventual loss of teeth. Approximately 50% of all adults in the United States over the age of thirty years have periodontitis.

Rheumatoid arthritis is a chronic, systemic, inflammatory disease that affects the synovial membranes of multiple joints in the body. Because the disease is systemic, there are many extra-articular features of the disease as well. For example, neuropathy, scleritis, lymphadenopathy, pericarditis, splenomegaly, arteritis, and rheumatoid nodules are frequent components of the disease. In most cases of rheumatoid arthritis, the subject has remissions and exacerbations of the symptoms. This disease is often associated with substantial loss of mobility due to pain and joint destruction. For example, about 60% of rheumatoid arthritis patients are unable to work ten years after the onset of their disease.

The mechanisms and pathways involved in mediating extensive degradation and remodeling of connective tissue in these inflammatory diseases or tumors are unclear. For example, a need exists for the identification of agents that inhibit the induction of connective tissue degrading enzymes and other inflammatory mediators. Such agents have potential for treating inflammatory diseases, such as atherosclerosis, periodontal disease, and rheumatoid arthritis, as well as tumors.

### SUMMARY OF THE DISCLOSURE

Connective tissue turnover can involve a series of proteases, such as matrix metalloproteinases (MMPs) and the plasminogen activation system. MMPs are zinc-binding endopeptidases that collectively degrade most of the components of the extracellular matrix. These enzymes have been linked to several diseases including tumors, rheumatoid arthritis, periodontal disease and atherosclerosis. The plasminogen activation system has been shown to be an important regulator of monocyte migration. Monocytes and macrophages are often located at chronic inflammatory lesion sites in which there is extensive degradation and remodeling of connective tissue. The plasminogen activation system and MMPs play a pivotal role in inflammatory diseases and tumor cell invasion, growth and metastasis.

The inventors have determined that plasmin regulates matrix metalloproteinase-1 (MMP-1) production in monocytes by binding to the annexin A2 heterotetramer. The inventors have also determined that inactive plasmin is an inhibitor of plasmin induction of MMP-1. Based on these observations, new methods of suppressing inflammation and tumors are disclosed, for example by using agents including inactive plasmin to inhibit plasmin-stimulated MMP-1 production.

In several embodiments, methods are provided for suppressing inflammation. The methods can include selecting the subject in need of suppression of inflammation and inhibiting plasmin activity in the subject to decrease MMP production, such as MMP-1 production, thereby suppressing the inflammation. Examples of inflammation include inflammation associated with a disease, including atherosclerosis, a periodontal disease, rheumatoid arthritis or a tumor (such a benign or malignant tumor). The methods can include administering to the subject an agent including a plasmin inhibitor (such as an irreversibly inactivated plasmin) at a therapeutically effective concentration to decrease MMP production. In an example, the agent interacts with an annexin A2 receptor inhibiting the ability of plasmin to bind to the annexin A2 receptor and stimulate MMP-1 production.

Methods are also provided herein to suppress a tumor. The methods can include selecting the subject in need of suppression of the tumor and administering to the subject an agent including inactive plasmin at a therapeutically effective concentration to decrease MMP production (e.g., MMP-1 production), thereby suppressing the tumor. In one example, the tumor is cancer. For example, the agent interacts with an annexin A2 receptor inhibiting the ability of plasmin to bind to the annexin A2 receptor and facilitate tumor cell invasion or metastasis. The method can also include administering one or more additional therapeutic agents, such as anti-neoplastic agents, at a therapeutically effective amount to the subject.

Also provided by the present disclosure are methods for modulating annexin A2 receptor activity. The methods can include contacting a cell with a therapeutically effective concentration of an agent including inactive plasmin, in which the inactive plasmin modulates the activity of an annexin A2 receptor and effects a change in the level of MMP production by the treated cell relative to MMP production in an untreated cell. In an example, inactive plasmin effects a change in the level of MMP-1. For example, the cell can be a tumor cell, such as a cancer cell, or a white blood cell.

In some embodiments, the inactive plasmin is administered to a subject who does not have a blood coagulation problem or who does not need thrombolysis or lysis of fibrin. In other examples, the inactive plasmin provides an anti-inflammatory or anti-tumor activity independent of interference with angiogenesis. In other examples, the plasmin is irreversibly (permanently) inactivated. For example, the plasmin is substantially free of enzymatic activity (including a substantial or complete reduction in the ability to proteolytically cleave fibrin or stimulate MMP production) under all conditions.

The foregoing and other features of the disclosure will become more apparent from the following detailed description of a several embodiments which proceeds with reference to the accompanying figures.

### BRIEF DESCRIPTION OF THE FIGURES

FIGS. 1A-1D illustrate the effect of high molecular weight-urokinase-type plasminogen activator (HMW-uPA), low molecular weight-urokinase-type plasminogen activator (LMW-uPA), and N-terminal fragment-urokinase-type plasminogen activator (ATF-uPA) on monocyte MMP-1 production. FIG. 1A is a digital image illustrating MMP-1 protein levels detected in monocytes by Western blot analysis in the presence or absence of lipopolysaccharide (LPS; 25 ng/ml) and the indicated concentrations of HMW-uPA. FIG. 1B is a digital image demonstrating MMP-1 protein levels detected in monocytes by Western blot analysis in which monocytes were obtained from three donors and cultured in the presence or absence of LPS and the indicated concentrations of LMW-uPA, HMW-uPA and ATF-uPA. FIG. 1C is a digital image illustrating MMP-1 mRNA levels determined by RT-PCR in monocytes that had been cultured in the presence or absence of LPS and the indicated concentrations of LMW-uPA, HMW-uPA and ATF-uPA. GAPDH served as an internal control for RNA levels. FIG. 1D includes a digital image and a graph illustrating MMP-1 mRNA levels determined by RT-PCR in monocytes after the addition of plasminogen activator inhibitor-1 (PAI-1) or HMW-uPA to control cultures or exposure of LPS-treated cultures to HMW-uPA or HMW-uPA (30 nM) that had been preincubated with PAI-1 (10 μg/ml)

FIGS. 2A-2C illustrate that uPA stimulation of MMP-1 production by activated monocytes is mediated through plasmin. FIG. 2A is a bar graph illustrating plasmin activity levels in monocytes cultured in 96 well plates in the presence or absence of LPS (25 ng/ml) and/or HMW-uPA (30 nM), LMW-uPA (30 nM), or ATF-uPA (30 nM). Plasmin activity was determined with a Spectrozyme PL kit (American Diagnostica Inc., Stamford, Conn.) with kinetic absorbance readings measured at 405 nM. FIG. 2B is a digital image illustrating MMP-1 protein detected by Western blot analysis in monocyte cultures following the addition of plasmin at the indicated concentrations in the presence or absence of LPS. FIG. 2C is a digital image demonstrating MMP-1 mRNA levels detected in monocytes determined by RT-PCR 8 hours after the addition of plasmin in the presence or absence of LPS. GAPDH served as an internal control for RNA levels.

FIGS. 3A-3F illustrate that antibodies against Annexin A2 (p36) or S100A10 (p11) block HMW-uPA or plasmin stimulated MMP-1 production. Monocytes were preincubated for 30 min with a goat antibody against annexin A2 (Gt anti-Ann A2), goat IgG (GtIgG) or the F(ab′)₂ portion of monoclonal antibodies against p36 (Annexin A2) or p11 (S100A10), a protein associated with annexin A2 involved in the formation of the heterotetramer. FIG. 3A is a digital image demonstrating MMP-1 protein levels detected by Western blot analysis following addition of LPS in the absence of HMW-uPA. FIG. 3B is a digital image illustrating MMP-1 protein levels detected by Western blot analysis following addition of LPS in the absence of plasmin. FIG. 3C is a histogram illustrating the specific binding of plasmin to annexin A2 and S100A10 as determined by a flow cytometry analysis with the addition of FITC-labeled plasmin to monocytes in the presence or absence of polyclonal antibodies against annexin A2. FIG. 3D is a digital image depicting MMP-1 protein levels detected by Western blot analysis following addition of LPS in the presence of HMW-uPA. FIG. 3E is a digital image illustrating MMP-1 protein levels detected by Western blot analysis following addition of LPS in the presence of plasmin. FIG. 3F is a histogram illustrating the specific binding of plasmin to annexin A2 and S100A10 as determined by flow cytometry analysis with the addition of FITC-labeled plasmin to monocytes in the presence goat IgG as isotype control or the F(ab′)₂ portion of monoclonal antibodies against p36 (Annexin A2) or p11 (S100A10).

FIGS. 4A-4C illustrate that stimulation of MMP-1 by HMW-uPA and plasmin is mediated in part by PGE₂. FIG. 4A is a digital image depicting cyclooxygenase-2 (COX-2) protein levels detected in monocyte cultures determined by Western blot analysis in the presence of HMW-uPA (30 nM) or plasmin (360 nM) in the absence or presence of LPS (25 ng/ml). β-actin protein levels were detected to serve as an indicator for equal sample loading. FIG. 4B is a bar graph illustrating media levels of PGE₂ measured by ELISA at 24 hours. FIG. 4C is a digital image illustrating MMP-1 protein levels detected by Western blot analysis in monocyte cultures following incubation with indomethacin for 30 minutes prior to the addition of LPS, HMW-uPA, plasmin or PGE₂ (1 μM).

FIGS. 5A and 5B illustrate ERK1/2 and p38 MAPK pathway involvement in HMW-uPA and plasmin stimulation of MMP-1 production by activated monocytes. FIG. 5A is a digital image illustrating MMP-1 protein levels in monocyte cultures determined by Western blot analysis following incubation for 30 minutes with 10 μM of PD98059 (PD), an inhibitor of ERK1/2, or 10 μM of SB203580 (SB), an inhibitor of p38 MAPK, prior to the addition of LPS, HMW-uPA or plasmin. FIG. 5B is digital image illustrating the phosphorylation levels of p38 MAPK or ERK1/2 detected by Western Blot analysis 1 hour after stimulation with LPS and compared with total p38 and ERK1/2 as a measure of equal sample loading.

FIGS. 6A-6D illustrate that inactive plasmin binds to annexin A2 blocking the binding of active plasmin and thereby inhibiting monocyte MMP-1 production. FIG. 6A is a digital image depicting MMP-1 protein levels detected by Western blot analysis in monocyte cultures following treatment with inactive plasmin (30 minutes) prior to the addition of LPS (25 ng/ml) or LPS plus plasmin. FIG. 6B is a bar graph illustrating plasmin activity measured with a Spectrozyme PL kit (American Diagnostica Inc., Stamford, Conn.) in a cell free assay to determine the effect of inactive plasmin on plasmin activity. Plasmin activity was also measured for the mixture of plasmin plus α2 anti-plasmin, which inhibits soluble plasmin, as a control for inhibition. FIG. 6C is a histogram illustrating the intensity of fluorescence detected in monocytes incubated with FITC-labeled plasmin alone, or pre-treated with inactive plasmin for 30 min then FITC-labeled plasmin by flow cytometry analysis. FITC-labeled bovine serum albumin served as a control for non-specific binding. FIG. 6D is a histogram illustrating the intensity of fluorescence detected in monocytes incubated with goat anti-human annexin A2, or pre-treated with inactive plasmin for 30 minutes and then goat anti-human annexin A2 (Gt anti-hu Ann A2). FITC-labeled anti-goat IgG was used as the secondary antibody and goat IgG served as a control for non-specific binding.

### DETAILED DESCRIPTION

### I. Terms and Abbreviations

ATF-uPA: N-terminal fragment-urokinase-type plasminogen activator

COX: cyclooxygenase

HMW-uPA: high molecular weight-urokinase-type plasminogen activator

LMW-uPA: low molecular weight-urokinase-type plasminogen activator

LPS: lipopolysaccharide

MAPK: mitogen-activated protein kinase

MMP-1: matrix metalloproteinase-1

PAI-1: plasminogen activator inhibitor

PMN: polymorphonuclear neutrophils

uPA: urokinase-type plasminogen activator

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

In order to facilitate review of the various embodiments of this disclosure, the following explanations of specific terms are provided:

Administer: To provide or give a subject an agent, such as inactive plasmin, by any effective route. Administration can be systemic or local. Exemplary routes of administration include, but are not limited to, oral, injection (such as subcutaneous, intramuscular, intradermal, intraperitoneal and intravenous), sublingual, rectal, transdermal (e.g., topical), intranasal, vaginal and inhalation routes. For example, if the chosen route is intravenous, the composition is administered by introducing the composition into a vein of the subject, and if the chosen route is intramuscular, the compositing is administered by introducing the composition in to a muscle. In particular examples, agents (such as those including inactive plasmin) are administered to a subject having or at risk of developing a chronic inflammatory disease or cancer. In one example, an agent including inactive plasmin is administered to a subject having a chronic inflammatory disease, such as atherosclerosis, rheumatoid arthritis, or periodontitis.

Agent: Any protein, nucleic acid molecule, compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional agent (such as an antinflammatory or antineoplastic agent, such as Etoposide, Doxorubicin, methotrexate, and Vincristine) induces the desired response (such as inducing a therapeutic or prophylactic effect when administered to a subject). In an example, an agent is inactive plasmin. In a particular example, an agent specifically inhibits plasmin-activation of annexin A2, thereby suppressing an inflammatory response.

Annexins: A family of structurally related eukaryotic proteins that reversibly bind membranes containing anionic phospholipids in a calcium-dependent manner. More than 160 annexins have been identified in different organisms, including mammals. The protein class is defined by its characteristic structure including a conserved core made up of four or eight domains of a 70 amino acid sequence forming five α-helices and a variable N-terminal region varying in length and amino acid sequence. The core domains harbor multiple calcium binding sites, which are all located on the convex side of the molecule. X-ray crystallographic analysis and mutagenesis studies have shown that the convex site is responsible for initial membrane binding. Calcium ions bound to these sites act as bridges connecting the protein with anionic lipid headgroups.

Annexin A2: A member of the annexin family, which is present in living cells as a monomer, heterodimer and heterotetramer. Monomeric annexin A2 is mainly located in the cytosol. The heterodimer is composed of two annexin A2 monomers and 3-phosphoglycerate-kinase. The most common form of annexin A2 is the heterotetrameric form, composed of two annexin A2 monomers and an 11 kilodalton (kDa) protein that is member of S100 family of calcium-binding proteins. This heterotetrameric complex (herein referred to as the annexin A2 receptor) can serve as a receptor for plasmin on monocytes. The annexin A2 receptor has been cloned, and nucleic acid and protein sequences are publicly available, for example from GenBank NM_{—}001014279.

Arthritis: Arthritis is an inflammation of the joints. Rheumatoid arthritis is an inflammatory disease that affects the synovial membranes of one or more joints in the body. It is the most common type of joint disease, and it is characterized by the inflammation of the joint. The disease is usually oligoarticular (affects few joints), but may be generalized. The joints commonly involved include the hips, knees, lower lumbar and cervical vertebrae, proximal and distal interphangeal joints of the fingers, first carpometacarpal joints, and first tarsometatarsal joints of the feet. Because the disease is systemic, there are many extra-articular features of the disease as well. For example, neuropathy, scleritis, lymphadenopathy, pericarditis, splenomegaly, arteritis, and rheumatoid nodules are frequent components of the disease. In most cases of rheumatoid arthritis, the subject has remissions and exacerbations of the symptoms. Rheumatoid arthritis is considered an autoimmune disease that is acquired and in which genetic factors appear to play a role.

Atherosclerosis: A disorder affecting arterial blood vessels. Atherosclerosis is a chronic inflammatory response in the walls of arteries, in large part due to plaque deposits. The plaque deposits often cause the artery to narrow and become more rigid, commonly referred to as a “hardening” or “furring” of the arteries. If the coronary arteries become narrow, blood flow to the heart can slow down or stop, causing chest pain (stable angina), shortness of breath, heart attack, and other symptoms. In an example, an agent including inactive plasmin is administered to a subject to suppress the chronic inflammatory response associated with atherosclerosis.

Binding: The ability of a first molecule to interact with a second molecule. In an example, the first molecule is an agent, such as an agent including inactive plasmin and the second molecule is a target molecule, such as annexin A2. In a particular example, an agent including inactive plasmin inhibits or reduces plasmin-binding to annexin A2. Binding affinity is the affinity of a molecule for its target. In one embodiment, affinity is calculated by a modification of the Scatchard method described by Frankel et al., Mol. Immunol., 16:101-106, 1979. In another embodiment, binding affinity is measured by a specific binding agent receptor dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, an agent including inactive plasmin is administered to a subject to suppress inflammation associated with cancer.

Clot lysis: A process referring to the breaking up of a thrombus or blood clot.

Contacting: Placement in direct physical association, including both a solid and liquid form. Contacting can occur in vitro with isolated cells or in vivo by administering to a subject. In an example, annexin A2 activity is modulated by contacting or exposing a cell, such as a white blood cell, with a therapeutically effective concentration of an agent, including inactive plasmin.

Disease: An abnormal condition of an organism that impairs bodily functions.

Inactive plasmin: A form of plasmin under conditions wherein the plasmin has a substantial or complete reduction in enzymatic activity, including a substantial or complete reduction in the ability to proteolytically cleave fibrin or stimulate MMP production. In an example, inactive plasmin is a form of plasmin in which the catalytic site has been irreversibly blocked with a peptide inhibitor. For example, the agent is inactive plasmin in that it has been prepared from Lys-plasmin by active site-specific inactivation with a Phe-Phe-Arg chloromethyl ketone. In another example, inactive plasmin is a form of plasmin in which the catalytic site includes a point mutation, such as a point mutation in the active site of the plasmin molecule. For example, the agent is inactive plasmin resulting from replacement of serine-741 to alanine in the active site. In a particular example, inactive plasmin is obtained from a commercial resource, such as Molecular Innovations, Inc. (Southfield, Mich.).

Inflammation: A localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, either systemically or locally at the site of inflammation. The inflammatory response may be measured by many methods well known in the art, such as the number of white blood cells, the number of polymorphonuclear neutrophils (PMN), a measure of the degree of PMN activation, such as luminal enhanced-chemiluminescence, or a measure of the amount of cytokines present. A primary inflammation disorder is a disorder that is caused by the inflammation itself. A secondary inflammation disorder is inflammation that is the result of another disorder. Inflammation can lead to a host of inflammatory diseases, such as rheumatoid arthritis, osteoarthritis, inflammatory lung disease (including chronic obstructive pulmonary lung disease), inflammatory bowl disease (including ulcerative colitis and Crohn's Disease), periodontal disease, polymyalgia rheumatica, atherosclerosis, systemic lupus erythematosus, systemic sclerosis, Sjogren's Syndrome, asthma, allergic rhinitis, and skin disorders (including dermatomyositis and psoriasis) and the like.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

Inhibit: To decrease, limit or block the action or function of a molecule. In an example, the activation of annexin A2 by plasmin is decreased, limited or block by inactive plasmin. For example, the inactive plasmin reduces activation of annexin A2 by plasmin inhibiting, reducing or decreasing MMP-1 production, such as a decrease of at least 10%, at least 20%, at least 50%, at least 70%, or even at least 90%.

Leukocytes: Cells in the blood, also termed “white blood cells,” that are involved in defending the body against infective organisms and foreign substances. Leukocytes are produced in the bone marrow. There are five main types of white blood cells, subdivided between two main groups: polymorphonuclear leukocytes (neutrophils, eosinophils, basophils) and mononuclear leukocytes (monocytes and lymphocytes). When an infection is present, the production of leukocytes increases.

Malignant: Cells which have the properties of anaplasia, invasion and metastasis.

Matrix metalloproteinases (MMPs): A family of zinc-dependent endopeptidases that act to modify or degrade the extracellular matrix. Each MMP contains a catalytic and pro-peptide regulatory domain and a variable number of carboxy-terminal hemopoexin-like structural domains and are broadly divided into subclasses based on substrate activity. Matrix metalloproteinase-1 (MMP-1) is a member of the metalloproteinase family, also referred to as collagenase-1 or interstitial collagenase. MMP-1 is a collagenase whose expression in vivo occurs in areas of rapid remodeling of the extracellular matrix under both normal physiological and pathological conditions. MMP-1 is expressed by several cell types including fibroblasts, keratinocytes, chondrocytes, monocytes, macrophages, hepatocytes and a variety of tumor cells (Westermarck and Kahari, Faseb J. 13: 781-792, 1999). Substrates for MMP-1 include collagens of type 1, II, III, VII, X, as well as large aggregating proteoglycans, serpins, and alpha-2-macroglobulin (Id.). The MMP-1 has been cloned from a variety of organisms, and nucleic acid and protein sequences are publicly available, for example from GenBank NM_{—}002421.2 and EMBL accession number: X58256.

In an example, plasmin stimulates MMP production, such as MMP-1 production, via modulation of annexin A2. MMP production is the synthesis or generation of one or more MMPs. In a particular example, an agent including inactive plasmin is administered to suppress inflammation or a tumor by effecting a change in MMP production by at least 10%, at least 20%, at least 50%, at least 70% or even at least 90%. A change, such as an increase or decrease, in MMP production can include a change in nucleic acid or protein expression of an MMP, such as MMP-1.

Modulate: To alter or induce a change in a cellular function, such as to cause an increase or a decrease in biological activity of a molecule. In a particular activity, an agent including inactive plasmin modulates annexin A2 (e.g., decreasing or inhibiting plasmin-mediated MMP production, such as MMP-1 production).

Neoplasm: Abnormal growth of cells, for example a tumor.

Normal cells: Non-diseased cells, such as non-tumor, non-malignant cells.

Periodontal disease: An inflammatory disease affecting the tissues that surround and support the teeth. Periodontitis is an inflammation of the periodontium, or one of the four tissues that support the teeth in the mouth, such as the gingival (gum tissue), cementum (outer layer of the roots of teeth), alveolar bone (bone sockets into which the teeth are anchored), and the periodontal ligaments (connective tissue fibres that connect the cementum and the gingiva to the alveolar bone). Periodontitis involves progressive loss of bone around teeth which may lead to loosening and eventual loss of teeth.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents, such as one or more compositions that include inactive plasmin.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Plasmin: A serine protease that includes, but is not limited to, Glu-plasmin, Lys-plasmin, derivatives, modified or truncated variants thereof. Active plasmin is plasmin under conditions where the plasmin is capable of proteolytically cleaving fibrin or activating matrix metalloproteinases (also referred to as plasmin activity). In a particular example, plasmin activity includes increasing MMP-1 production or synthesis, such as by modulating annexin A2. A plasmin inhibitor is a molecule that inhibits or reduces the activity of plasmin, including the proteolytic cleaving of fibrin or activation of MMPs. In an example, a plasmin inhibitor is irreversibly-inactivated plasmin. Plasmin inhibitors can also include those well known in the art including Aprotinin, alpha 2 anti-plasmin and a recombinant form of tissue factor pathway inhibitor-2 (TFPI-2; in which the Kunitz-type domain (KD1) has been mutated).

Sample: A biological specimen that contain cells, genomic DNA, RNA (including mRNA), protein or combinations thereof, obtained from a subject. Examples include, but are not limited to, peripheral blood or a subcomponent thereof such as serum or plasma, urine, saliva, tissue biopsy, surgical specimen, fine needle aspirate, and autopsy material. In a particular example, a sample is or includes monocytes obtained from a subject having or suspected of having cancer, atherosclerosis, a periodontal disease or rheumatoid arthritis.

Subject: Living multi-cellular vertebrate organisms, a category that includes human and non-human mammals (such as laboratory or veterinary subjects). In an example, a subject is a human. In an additional example, a subject is selected that is in need of suppressing inflammation or a tumor. For example, the subject is either at risk of developing an inflammation or tumor or has an inflammation or tumor in need of treatment.

Suppress (or decrease): To reduce the quality, amount, or strength of something. In one example, a therapy suppresses or reduces inflammation or one or more symptoms associated with inflammation, for example as compared to the response in the absence of the therapy. In a particular example, a therapy suppresses the inflammation by at least 10%, at least 20%, at least 50%, at least 70%, or even at least 90%. Such suppression can be measured using methods disclosed herein.

In another example, a therapy suppresses a tumor (such as the size of a tumor, the number of tumors, the metastasis of a tumor, or combinations thereof), or one or more symptoms associated with a tumor, for example as compared to the response in the absence of the therapy. In a particular example, a therapy suppresses the size of a tumor, the number of tumors, the metastasis of a tumor, or combinations thereof, subsequent to the therapy, such as a decrease of at least 10%, at least 20%, at least 50%, at least 70% or even at least 90%. Such decreases can be measured using the methods disclosed herein.

Therapeutically effective amount: An amount of an agent (such as an agent that includes inactive plasmin), that alone, or together with one or more additional therapeutic agents (such anti-inflammatory or antineoplastic agents), induces the desired response, such as treatment of inflammation or a tumor, such as cancer. In one example, it is an amount of an agent including inactive plasmin needed to prevent or delay the development of inflammation or a tumor, prevent or delay the metastasis of a tumor, cause regression of an existing inflammation or tumor, or treat one or more signs or symptoms associated with an inflammation or a tumor, in a subject. Ideally, a therapeutically effective amount provides a therapeutic effect without causing a substantial cytotoxic effect in the subject. The preparations disclosed herein are administered in therapeutically effective amounts.

In an example, a desired response is to reduce or decrease inflammation associated with an inflammatory disease, such as atherosclerosis, periodontitis, or rheumatoid arthritis. For example, the agent can decrease the inflammation by a desired amount, for example by at least 5%, at least 10%, at least 15%, at least 20%, at least 25%, at least 30%, at least 50%, at least 75%, or even at least 90%, as compared to a response in the absence of the agent.

In one example, a desired response is to decrease the size, volume, or number (such as metastases) of a tumor that overexpresses MMP-1. For example, the agent can decrease the size, volume, or number of tumors by a desired amount, for example by at least 5%, at least 10%, at least 15%, at least 20%, at least 25%, at least 30%, at least 50%, at least 75%, or even at least 90%, as compared to a response in the absence of the agent.

The effective amount of an agent that includes inactive plasmin, that is administered to a human or veterinary subject will vary depending upon a number of factors associated with that subject, for example the overall health of the subject. An effective amount of an agent can be determined by varying the dosage of the product and measuring the resulting therapeutic response, such as the regression of a tumor or suppression of inflammation. Effective amounts also can be determined through various in vitro, in vivo or in situ immunoassays. The disclosed agents can be administered in a single dose, or in several doses, as needed to obtain the desired response. However, the effective amount of can be dependent on the source applied, the subject being treated, the severity and type of the condition being treated, and the manner of administration.

In particular examples, a therapeutically effective dose of an agent including inactive plasmin includes at least 1 μg daily (such as 1-100 μg or 5-50 μg) if administered via injection, or at least 1 mg daily if administered topically (such as 1-100 mg or 5-50 mg) of the agent that includes inactive plasmin. In particular examples, such daily dosages are administered in one or more divided doses (such as 2, 3, or 4 doses) or in a single formulation.

The disclosed agents that include inactive plasmin can be administered alone, in the presence of a pharmaceutically acceptable carrier, in the presence of other therapeutic agents (such as other anti-neoplastic agents or anti-inflammatory agents), or both.

Treated cell: A cell that has been contacted with a desired agent in an amount and under conditions sufficient for the desired response. In one example, a treated cell is a cell that has been exposed to inactive plasmin under conditions sufficient for the inflammation or tumor to be suppressed.

Treating or treatment: Refers to a therapeutic intervention that ameliorates a sign or symptom of a disease or pathological condition related to a disease (such as, atherosclerosis, a periodontal disease, rheumatoid arthritis or a tumor, for example cancer). Treatment can also induce remission or cure such condition. In particular examples, treatment includes inhibiting a tumor, for example by inhibiting the full development of a tumor, such as preventing development of a metastasis or the development of a primary tumor. Inhibition does not require a total absence of a tumor. In other examples, treatment includes inhibiting or reducing inflammation.

Reducing or suppressing a sign or symptom associated with a disease (such as, atherosclerosis, a periodontal disease, rheumatoid arthritis or a tumor, for example cancer) can be evidenced, for example, by a delayed onset of clinical symptoms of the disease in a susceptible subject (such as a subject having a tumor which has not yet metastasized), a reduction in severity of some or all clinical symptoms of the disease, a slower progression of the disease (for example by prolonging the life of a subject having the disease), a reduction in the number of relapses of the disease, an improvement in the overall health or well-being of the subject, or by other parameters well known in the art that are specific to the particular disease.

Tumor: A neoplasm that may be either benign or malignant. In an example, a tumor is cancer. In specific examples, an agent including inactive plasmin is administered to suppress a tumor, for example a malignant tumor, such as by suppressing tumor cell invasion or metastasis.

Under conditions sufficient for: A phrase that is used to describe any environment that permits the desired activity. In one example, includes administering a therapeutically effective amount of a composition that includes inactive plasmin, sufficient to allow the desired activity. In particular examples the desired activity is suppressing inflammation or a tumor, such as cancer.

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of a disease, for example atherosclerosis, a periodontal disease, rheumatoid arthritis or a tumor (e.g., cancer)

Untreated cell: A cell that has not been contacted with a desired agent, such as a test agent. In an example, an untreated cell is a cell that receives the vehicle in which the desired agent was delivered.

### II. Methods of Suppressing Inflammation

It is shown herein that plasmin regulates MMP-1 production in monocytes by binding to the annexin A2 heterotetramer. It is also demonstrated herein that inactive plasmin inhibits plasmin stimulation of MMP-1 production in such cells. In one example, inactive plasmin inhibits plasmin-stimulated MMP-1 production by inhibiting the binding of plasmin to the annexin A2. Based on these observations, new methods of suppressing inflammation are disclosed, for example by using agents including inactive plasmin to inhibit plasmin-stimulated MMP-1 production.

Methods of suppressing inflammation are disclosed by selecting a subject in need of suppression of inflammation and inhibiting plasmin activity in the subject to decrease MMP production, such as MMP-1 production, thereby suppressing the inflammation. In some examples, subjects are initially screened to determine if they have increased levels of MMP-1 in their serum, whether they have a disease associated with increased expression of MMP-1, or combinations thereof. For example, the diagnostic methods known to those of skill in the art, including immunodetection techniques, can be used to screen subjects to determine if they are candidates for the disclosed therapies.

The method can suppress inflammation either in vitro or in vivo. When suppressing inflammation in vivo, the agent can be used to either avoid inflammation or to treat an existing inflammation. The inflammation may be a primary inflammatory disorder, meaning that it is not secondary to another disorder that causes inflammation as a consequence of the primary disorder. For example, a myocardial infarction or thromboembolus may result in secondary inflammation. The plasmin may be irreversibly inactive to help avoid unwanted thrombolytic effects of the agent, such as systemic degradation of fibrin. In an example, the plasmin is substantially free of enzymatic activity, including a substantial or complete reduction in the ability to proteolytically cleave fibrin or stimulate MMP production, under all conditions.

The disclosed methods can be used to suppress inflammation associated with a disease, such as atherosclerosis, periodontal disease, rheumatoid arthritis or a tumor. The inflammation may be musculoskeletal, neurological, cardiovascular, urological, gynecological, ophthalmic, dental, gastrointestinal, otological, dermatologic or respiratory. Suppression of inflammation can include delaying the development of inflammation in a subject. Treatment of inflammation also includes reducing signs or symptoms associated with the presence of inflammation. Such inhibition can in some examples decrease or suppress inflammation by at least 10% (such as by at least 20%, at least 50%, or at least 90%) as compared to a response in the absence of the agent including inactive plasmin. For example, inflammation associated with a benign tumor can be suppressed by at least 10% (such as by at least 20%, at least 50%, or at least 90%) as compared to inflammation in the absence of the treatment. The methods disclosed herein can also be used to suppress or inhibit inflammation associated with malignant tumors (such as cancer). In other examples, treatment using the methods disclosed herein prolongs the time of survival of the subject.

In one example, inactive plasmin inhibits plasmin-stimulated MMP-1 production by inhibiting the binding of plasmin to the annexin A2 receptor. In other examples, inactive plasmin regulates cell functions, such as stimulation of MMP-1 production, by binding to sites other than annexin A2 receptor.

The methods can include administering an agent including a therapeutically effective amount of inactive plasmin. Additional agents can also be administered to the subject, such as anti-inflammatory agents, in combination with the agent including inactive plasmin.

### III. Methods of Suppressing a Tumor

Methods are disclosed herein for treating tumors, such as those associated with increased MMP-1 activity. Methods for suppressing a tumor can include selecting a subject in need of suppression of a tumor and inhibiting plasmin activity in the subject to decrease MMP production, thereby suppressing the tumor. In one example, increased expression of MMP-1 can be detected in serum or plasma obtained from a subject having such a tumor. For example, detection of MMP-1 in the serum of a subject (for example at a level of at least twice that found in a subject not having a tumor), detection of increased levels of MMP-1 in the tumor (for example relative to expression of MMP-1 in adjacent non-tumor cells), or both, indicates that the subject can benefit from the disclosed methods.

In some examples, subjects are initially screened to determine if they have increased levels of MMP-1 in their serum, whether they have a tumor that has increased expression of MMP-1 (for example relative to adjacent non-tumor cells), or combinations thereof. For example, the diagnostic methods known to those of skill in the art, including immunodetection techniques, can be used to screen subjects to determine if they are candidates for the disclosed therapies.

In some examples, the tumor is treated in vivo, for example in a mammalian subject, such as a human subject. A tumor is an abnormal growth of tissue that results from excessive cell division. A particular example of a tumor is cancer. For example, the current application is useful for the treatment (such as the inhibition or suppression of metastasis) of tumors (such as cancer). Exemplary tumors that can be treated using the disclosed methods include, but are not limited to cancers of the head and neck, oral cavity, breast, lung, skin, esophagus, colon, stomach and ovaries, including metastases of such tumors.

Suppression of a tumor can include inhibiting or delaying the development of the tumor in a subject (such as inhibiting metastasis of a tumor), and also includes reducing signs or symptoms associated with the presence of such a tumor (for example by reducing the size or volume of the tumor or a metastasis thereof). In a specific example, treatment includes reducing the growth of cells of the tumor, or even killing the tumor cells (for example by causing the cells to undergo apoptosis). Such reduced growth can in some examples decrease or slow metastasis of the tumor, or reduce the size or volume of the tumor. In one example, treatment of a tumor includes reducing the invasive activity of the tumor in the subject, for example by reducing the ability of the tumor to metastasize. In certain examples, metastasis is reduced by at least 10% (such as at least 20%, at least 50%, or at least 90%), for example as compared to an amount of metastasis in the absence of the agent including inactive plasmin.

In particular examples, the method includes administering to the subject a therapeutically effective amount of an agent including inactive plasmin that reduces cellular invasion resulting from the interaction between plasmin and annexin A2 receptor, thereby suppressing the tumor. In an example, cellular invasion is reduced by at least 10% (such as at least 20%, at least 50%, or at least 90%), for example as compared to an amount of cellular invasion in the absence of the agent including inactive plasmin. In some examples, treatment using the methods disclosed herein prolongs the time of survival of the subject.

### IV. Methods of Modulating Annexin A2 Receptor Activity

Methods are provided herein for modulating annexin A2 receptor activity, such as by administering agents that effect a change in the level of MMP production in a cell. In one example, the method includes contacting at least one cell with an agent including inactive plasmin. In one example, the cell expresses an annexin A2 receptor. For example, the cell can be a tumor cell, such as a cancer cell, or a white blood cell. The inactive plasmin modulates the activity of an annexin A2 receptor and effects a change in the level of MMP production by the treated cell relative to MMP

A change, such as an increase or decrease, in MMP production can include a change in mRNA or protein expression of an MMP, such as MMP-1. In a particular example, a change includes a decrease or reduction by at least 10% (such as by at least 20%, at least 50% or at least 90%) in mRNA or protein expression of MMP-1. For example, RT-PCR can be employed to compare MMP-1 mRNA expression levels in the presence and absence of the agent including inactive plasmin. In other examples, immunodetection techniques, such as ELISA, can be utilized to compare MMP-1 protein levels in the presence and absence of the agent including inactive plasmin.

### Therapeutic Agents

Therapeutic agents are agents that when administered in therapeutically effective amounts induce the desired response (e.g., suppression of inflammation or a tumor). The methods can include administering an agent including a therapeutically effective amount of inactive plasmin. In an example, the inactive plasmin is a form of plasmin in which the catalytic site has been irreversibly blocked with a peptide inhibitor. For example, the agent is inactive plasmin that has been prepared from Lys-plasmin by active site-specific inactivation with a Phe-Phe-Arg chloromethyl ketone. In another example, inactive plasmin is a form of plasmin which the catalytic site includes a point mutation, such as a point mutation in the active site of the plasmin molecule. For example, the agent is inactive plasmin resulting from replacement of serine at position 741 by alanine in the active site. Additional agents can also be administered to the subject, such as anti-inflammatory or anti-neoplastic agents, in combination with disclosed therapeutic agents.

### Screening Subjects

Subjects can be screened prior to initiating the disclosed therapies, for example to select a subject in need of suppression of inflammation or a tumor. In an example, a subject in need of the disclosed therapies is selected by determining the level of MMP-1 production in a biological sample. The detection of increased levels of MMP-1 in a subject with or at risk of developing an inflammation, indicates that the inflammation can be treated using the methods provided herein. Moreover, the presence of a tumor that overexpresses MMP-1 indicates that the tumor can be treated using the disclosed methods.

In one example, the biological sample (such as a serum or biopsy sample) is analyzed using immunodetection methods. For example, the biological sample can be incubated with an antibody that specifically binds to MMP-1. The primary antibody can include a detectable label. For example, the primary antibody can be directly labeled, or the sample can be subsequently incubated with a secondary antibody that is labeled (for example with a fluorescent label). The label can then be detected, for example by microscopy, ELISA, flow cytometry, or spectrophotometry. In another example, the biological sample is analyzed by Western blotting for the presence of MMP-1. In one example, a subject is screened by determining whether increased levels of MMP-1 are present in the subject's serum (for example relative to a level present in a serum sample from a subject not having an inflammatory disease or tumor), for example using an antibody that specifically binds MMP-1.

As an alternative to analyzing the sample for the presence of proteins, the presence of nucleic acids can be determined. For example, the biological sample can be incubated with primers under conditions that permit the amplification of MMP-1. Exemplary methods include RT-PCR. In another example, the biological sample is incubated with probes that can bind to MMP-1 nucleic acid (such as cDNA, genomic DNA, or RNA (such as mRNA)) under high stringency conditions. The resulting hybridization can then be detected using methods known in the art. In one example, a subject is screened by determining whether they have increased levels of MMP-1 mRNA present in the subject's serum (for example relative to a level present in a sample from a subject not having an inflammatory disease or tumor or a control value determined to be indicative of the mRNA level present in non-diseased cells).

### Administration

Methods of administration of the disclosed agents are routine, and can be determined by a skilled clinician. For example, the disclosed agents (such as those that include inactive plasmin) can be administered via injection, orally, topically, transdermally, parenterally, or via inhalation or spray. In a particular example, an agent including inactive plasmin is administered intravenously to a mammalian subject, such as a human.

The therapeutically effective amount of the agents administered can vary depending upon the desired effects and the subject to be treated. In one example, the method includes daily administration of at least 1 μg of a therapeutic agent to the subject (such as a human subject). For example, a human can be administered at least 1 μg or at least 1000 mg of the agent daily, such as 10 μg to 100 μg daily, 100 μg to 1 mg daily, 100 μg to 1000 mg for example 100 μg daily, 1 mg daily, 10 mg daily, 100 mg daily, or 1000 mg. In an example, the subject is administered at least 1 μg (such as 1-100 μg) intravenously of the therapeutic agent (such as an agent including inactive plasmin). In one example, the subject is administered at least 1 mg intramuscularly (for example in an extremity) of such composition. The dosage can be administered in divided doses (such as 2, 3, or 4 divided doses per day), or in a single dosage daily. In a specific example, the subject is administered at least 0.15 mg per kg of body weight of the agent approximately every four weeks for at least 6 months. For example, 0.15 mg/kg, 0.5 mg/kg, 2 mg/kg, 3 mg/kg, 5 mg/kg or 6 mg/kg is administered, such as via intravenous or subcutaneous injections, every 28 days for 6 months.

In particular examples, the subject is administered the agent that includes inactive plasmin on a multiple daily dosing schedule, such as at least two consecutive days, 10 consecutive days, and so forth, for example for a period of weeks, months, or years. In one example, the subject is administered the agent daily for a period of at least 30 days, such as at least 2 months, at least 4 months, at least 6 months, at least 12 months, at least 24 months, or at least 36 months.

In specific examples, the agent for administration can include a solution of the disclosed agents including inactive plasmin dissolved in a pharmaceutically acceptable carrier, such as an aqueous carrier. A variety of aqueous carriers can be used, for example, buffered saline and the like. These solutions are sterile and generally free of undesirable matter. These agents may be sterilized by conventional, well known sterilization techniques. The agents may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of inactive plasmin in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of the agent including inactive plasmin per subject per day. Dosages from 0.1 up to about 100 mg per subject per day may be used. Actual methods for preparing administrable agents will be known or apparent to those skilled in the art and are described in more detail in such publications as Remington's Pharmaceutical Science, 19th ed., Mack Publishing Company, Easton, Pa. (1995).

The disclosed agents including inactive plasmin may be provided in lyophilized form and rehydrated with sterile water before administration, although they are also provided in sterile solutions of known concentration. The agent solution is then added to an infusion bag containing 0.9% Sodium Chloride, USP, and typically administered at a dosage of from 0.5 to 15 mg/kg of body weight. Considerable experience is available in the art in the administration of compounds such as inactive plasmin. These drugs can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The disclosed agents including inactive plasmin can further include one or more biologically active or inactive compounds (or both), such as anti-inflammatory or anti-neoplastic agents and conventional non-toxic pharmaceutically acceptable carriers, respectively. Examples of such biologically inactive compounds include, but are not limited to: carriers, thickeners, diluents, buffers, preservatives, and carriers. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

Controlled release parenteral formulations can be made as implants, oily injections, or as particulate systems. For a broad overview of protein delivery systems see, Banga, Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, ed., Marcel Dekker, Inc., New York, N.Y., pp. 219-342 (1994); and Tice & Tabibi, Treatise on Controlled Drug Delivery, ed., Marcel Dekker, Inc. New York, N.Y., pp. 315-339, (1992) both of which are incorporated herein by reference.

Polymers can be used for ion-controlled release of the agents disclosed herein. Various degradable and nondegradable polymeric matrices for use in controlled drug delivery are known in the art (Langer, Accounts Chem. Res. 26: 537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res., 9: 425-434, 1992; and Pec et al., J. Parent. Sci. Tech., 44(2): 58-65, 1990). Alternatively, hydroxyapatite has been used as a microcarrier for controlled release of proteins (Ijntema et al., Int. J. Pharm., 112: 215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

The plasmin inhibitors can also be administered with anti-tumor pharmaceutical treatments, which can include radiotherapeutic agents, anti-neoplastic chemotherapeutic agents, antibiotics, alkylating agents and antioxidants, kinase inhibitors, and other agents. These treatments can be administered either concurrently (for example in a single composition with the plasmin inhibitors) or separately. Particular examples of additional therapeutic agents can that can be used include microtubule binding agents, DNA intercalators or cross-linkers, DNA synthesis inhibitors, DNA and/or RNA transcription inhibitors, antibodies, enzymes, enzyme inhibitors, gene regulators, and angiogenesis inhibitors. These agents (which are administered at a therapeutically effective amount) and treatments can be used alone or in combination (with one another and/or with the plasmin inhibitors). Methods and therapeutic dosages of such agents are known to those skilled in the art, and can be determined by a skilled clinician.

“Microtubule binding agent” refers to an agent that interacts with tubulin to stabilize or destabilize microtubule formation thereby inhibiting cell division. Examples of microtubule binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

Suitable DNA and/or RNA transcription regulators, including, without limitation, actinomycin D, daunorubicin, doxorubicin and derivatives and analogs thereof also are suitable for use in combination with the disclosed therapies.

DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors include, without limitation, camptothecin, etoposide, formestane, trichostatin and derivatives and analogs thereof.

Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

“Angiogenesis inhibitors” include molecules, such as proteins, enzymes, polysaccharides, oligonucleotides, DNA, RNA, and recombinant vectors, and small molecules that function to reduce or even inhibit blood vessel growth. Angiogenesis is implicated in most types of human solid tumors. Angiogenesis inhibitors are known in the art and examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof.

Kinase inhibitors include Gleevec®, Iressa®, and Tarceva™ that prevent phosphorylation and activation of growth factors.

Antibodies that can be used include Herceptin and Avastin that block growth factors and the angiogenic pathway.

Among various uses of the agents disclosed herein are disease conditions associated with inflammation (e.g., atherosclerosis, a periodontal disease, rheumatoid arthritis or cancer), or a tumor, such as cancer.

The following examples are provided to illustrate certain particular features and/or embodiments. These examples should not be construed to limit the invention to the particular features or embodiments described.

### EXAMPLES

### Example 1

### Materials and Methods for Characterizing Plasmin-Annexin A2 Induction of MMP-1 Production

This example provides the materials and methods utilized for characterizing plasmin-annexin A2 induction of MMP-1 production in monocytes.

Reagents. High molecular weight urokinase plasminogen activator (HMW-uPA), low molecular weight uPA (LMW-uPA), amino-terminal fragment-uPA (ATF-uPA) and α2-antiplasmin were obtained from American Diagnostica Inc. (Stamford, Conn.). Plasmin (greater than or equal to 3 units/mg protein) was purchased from Sigma-Aldrich (St. Louis, Mich.) and inactive plasmin and a stable mutant form of human PAI-1 were from Molecular Innovations, Inc. Annexin A2 polyclonal antibody was obtained from Santa Cruz Biotechnology (Santa Cruz, Calif.) and monoclonal antibodies against annexin A2 and F(ab′)₂ antibodies against annexin A2 (p36) and S100A10 (p11) were from Becton Dickinson Biosciences (Franklin Lakes, N.J.). LPS Escherichia coli 05:B55 was purchased from Sigma-Aldrich (St. Louis, Mo.). ERK1/2/phospho-ERK1/2 and p38/phospho-p38 antibodies were from Cell Signaling Technology (Beverly, Mass.). MMP-1 antibodies used were rabbit polyclonal antibodies against MMP-1 (provided by Dr. Henning Birkedal-Hansen, NIDCR/NIH) and a mouse anti-human MMP-1 monoclonal antibody (Chemicon International, Inc., Temecula, Calif.). Rabbit anti-COX-2 antibodies were obtained from Cayman Chemical Company (Ann Arbor, Mich.) and mouse anti-β-actin antibodies were from Chemicon International, Inc., Temecula, Calif.).
Purification and culture of human monocytes. Human peripheral blood cells were obtained by leukapheresis of non-disease subjects. The monocyte fraction was purified by counter-flow centrifugal elutriation as previously described (Wahl et al., Cell Immunol. 85: 373-383, 1984; and Wahl et al., “In Current Protocols in Immunology,” eds. John Wiley & Sons, Inc. NY. Vol. 2: 7.6A.1-7.6A.10., 2005) and contained greater than 90% monocytes as determined by morphology and flow cytometry. Monocytes were cultured in serum-free Dulbecco Modified Eagle's Medium (DMEM; Cambrex, Walkersville, Md.) supplemented with 2 mM L-glutamine (Mediatech, Herndon, Va.) and 10 μg/ml gentamycin (Cambrex, Walkersville, Md.).
Western blot analysis of MMP-1, MAPKs and COX-2. For MMP-1 determination, purified monocytes were cultured at a density of 5×10⁶/ml of DMEM in 12 well polystyrene plates (Corning Incorporated Life Sciences, Lowell, Mass.). After 36 to 48 hours of treatment with reagents, the conditioned media were centrifuged and collected. Bovine serum albumin (BSA; 40 μg/ml) was added to the culture supernatants prior to the precipitation of the proteins with cold ethanol (final concentration, 60%) for at least 15 min at −70° C. The proteins were pelleted by microcentrifuging at 20,800×g for 12 min, washed with ethanol, and lyophilized by rotary evaporation. The lyophilized proteins were resuspended in sodium dodecyl sulfate (SDS)-Laemmli buffer [500 mM Tris-HCl (pH 6.8)/10% SDS/0.01% bromophenol-blue/20% glycerol], reduced with 5% β-mercaptoethanol, heated for 4 min at 100° C., and electrophoresed on a 10 or 12% Tris-glycine gel in SDS running buffer [25 mM Tris-HCl (pH 8.3)/192 mM glycine/10% SDS]. The proteins were transferred onto 0.45 μm nitrocellulose in a buffer containing 25 mM Tris-HCl (pH 8.3)/192 mM glycine/20% methanol and blocked with 50 mM Tris-HCl (pH 7.5)/150 mM NaCl (TBS) containing 5% nonfat dry milk for at least 1 hour. The blots were then incubated overnight with antibodies against MMP-1.

For detection of the levels of activated ERK1/2 and p38 MAPKs monocytes were cultured in DMEM at 5×10⁶/ml in suspension. Ten to 60 minutes after the addition of reagents, the cells were pelleted and lysed with SDS loading buffer (prepared as described above). The supernatants were loaded onto 12% Tris-glycine gels. The blots were then incubated overnight with mouse anti-human antibodies against the phosphorylated forms of ERK1/2 and p38 and with rabbit anti-human antibodies against total ERK1/2 and p38 as a measure of equal loading of the gels.

Cell protein isolation for the determination of COX-2 protein levels were prepared as previously described (Zhang et al., J. Clin. Invest. 99: 894-900, 1997). Briefly, 20×10⁶ monocytes in 4 ml of DMEM were cultured in suspension in 17 ml polypropylene tubes overnight in the presence or absence of reagents. The cells were then washed in phosphate buffered saline with protease inhibitors. The cell pellets were resuspended in 250 mM sucrose containing protease inhibitors and sonicated (Ultrasonic Cell Disrupter, Kontes, American Instrument Exchange, Inc., Haverhill, Mass.). Nuclear debris and unbroken cells were pelleted at 100×g and the supernatant microfuged at 1,500×g to pellet cell membrane proteins. Equal amounts of protein were loaded onto 12% Tris Glycine gels. The blots were incubated overnight with rabbit anti-COX-2 antibodies. Equal loading of samples was measured with mouse anti-β-actin antibodies.

Western blots for MMP-1, MAPKs and COX-2 were incubated overnight with primary antibodies, washed and analyzed by the addition of Alexa Fluor 680 goat anti-rabbit or Alexa Fluor 750 goat anti-mouse antibodies (Molecular Probes® Inc., Eugene, Oreg.) and the infrared fluorescence detected with the Odyssey infrared imaging system (LI-COR, Lincoln, Nebr.). Densitometry analysis of the bands on the Western blots was determined with the LI-COR software analysis program or the ImageQuant software analysis program (Amersham Biosciences, Piscataway, N.J.).

Plasmin activity assay. Plasmin activity was determined with the SPECTROZYME PL kit (American Diagnostica Inc., Stamford, Conn.) which utilizes the chromogenic substrate H-D-norleucyl-hexahydrotyrosol-lysine-para-nitroanilide diacetate. For detection of cell-associated plasmin activity, monocytes were cultured at 1×10⁶/well in Hanks Balanced Solution containing calcium and magnesium (Hyclone, Logan, Utah) with the plasmin substrate. Kinetic absorbance readings were measured at 405 nm.
RT-PCR. Total cellular RNA was extracted with the RNeasy Mini Kit (QIAGEN® Inc., Valencia, Calif.) 8 hours after stimulation of monocytes with LPS. Transcript levels of MMP-1 were determined using semiquantitative reverse transcriptase-polymerase chain reaction (RT-PCR) with GADPH as an internal control. The primer sets for MMP-1 were 5′-TGTGGTGTCTCACAGCTTCC-3′ (SEQ ID NO:1) and 5′-CACATCAGGCACTCCACATC-3′ (SEQ ID NO:2) and for GAPDH 5′-TCGGAGTCAACGGATTTGGTCGTA-3′ (SEQ ID NO:3) and 5′-ATGGACTGTGGTCATGAGTCC-3′ (SEQ ID NO:4). OneStep RT-PCR kit (QIAGEN® Inc., Valencia, Calif.) was used with the following reaction components: 5 μl 5× OneStep RT-PCR buffer, 10 mM dNTP, 10 μM MMP-1 primer mix, 6 μM GAPDH primer mix, 0.5 μg RNA template, 10 μl OneStep RT-PCR enzyme mix, and RNase-free water were added for a total of 25 μl. PCR times were: 30 min at 50° C. for reverse transcription, 15 min at 95° C. for initial PCR, 36 cycles of 40 s at 94° C., 45 s at 57° C., and 1 min at 72° C.; and 10 min at 72° C. for the final extension. The amplified DNA was separated by 1.7% agarose gel electrophoresis, stained with SYTO 60 red fluorescent nucleic acid stain (Molecular Probes®, Eugene, Oreg.), and the intensity of the stained bands was analyzed with an infrared imaging system.
Cell staining and flow cytometry analysis. Plasmin and BSA were FITC-conjugated, as previously described (Zhou et al., Clin. Exp. Immunol. 137:88-100, 2004), using a FluoroTag™ FITC conjugation kit (Sigma-Aldrich, St. Louis, Mo.). Purified human monocytes were fixed and permeablized (buffers from eBiosciences, Inc., San Diego, Calif.). Three to 10×10⁵ cells were preincubated with 5 μg human whole IgG (Jackson ImmunoResearch Laboratories, Inc., West Grove, Pa.) in 90 μl PBS containing 0.5% BSA and 2 mM EDTA at 4° C. for 30 min to minimize the effect of non-specific FcR binding sites. FITC-conjugated BSA, plasmin (2.5 μg in 10 μl) or anti-human antibodies (1-2.5 μg/10 μl) then were added, respectively, incubated for 30 min and washed and analyzed by the FACSCalibur system (Becton Dickinson Biosciences, Franklin Lakes, N.J.). For annexin A2 binding site blocking experiments, polyclonal goat anti-human annexin A2 IgG (Santa Cruz Biotechnology, Santa Cruz, Calif.), mouse anti-human annexin A2 (p36) IgG F(ab′)₂, mouse anti-human S100A10 (p11) IgG F(ab′)₂ (Becton Dickinson Biosciences, Franklin Lakes, N.J.), and inactive-plasmin (10 μg/ml), respectively, were preincubated with the cells at 4° C. for 30 min before the addition of 2.5 μg/ml of plasmin-FITC. For plasmin-FITC binding to monocytes, an equal amount of BSA-FITC served as negative control. Plasmin and inactive-plasmin were also pre-incubated with monocytes to block the binding sites of annexin A2 recognized by anti-human annexin A2 antibodies. For indirect immuno-staining polyclonal goat anti-human annexin A2 IgG (Jackson ImmunoResearch Inc., West Grove, Pa.) or goat IgG, as negative control, was added to the cells followed by the addition of FITC-labeled rabbit anti-goat IgG F(ab′)₂ as secondary antibody. Flow cytometry results were analyzed by CELLQuest software (Becton Dickinson Biosciences, Franklin Lakes, N.J.).
Statistical analysis. Comparison between group means was analyzed using ANOVA. The statistic data represent the mean±SEM. A value of P<0.01 was regarded as significant.

### Example 2

### Catalytically Active Urokinase-Type Plasminogen Activator Stimulates MMP-1 Production

This example demonstrates that catalytically active urokinase-type plasminogen activator (uPA) enhances MMP-1 production by activated monocytes

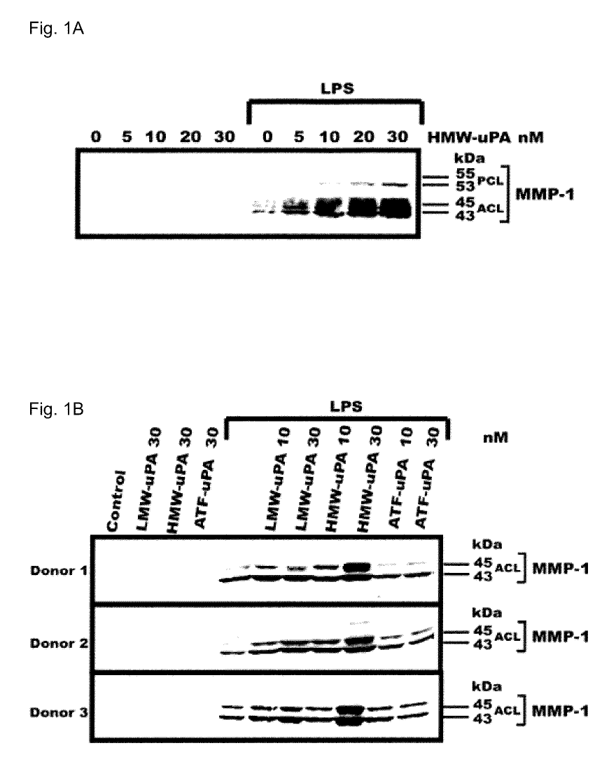

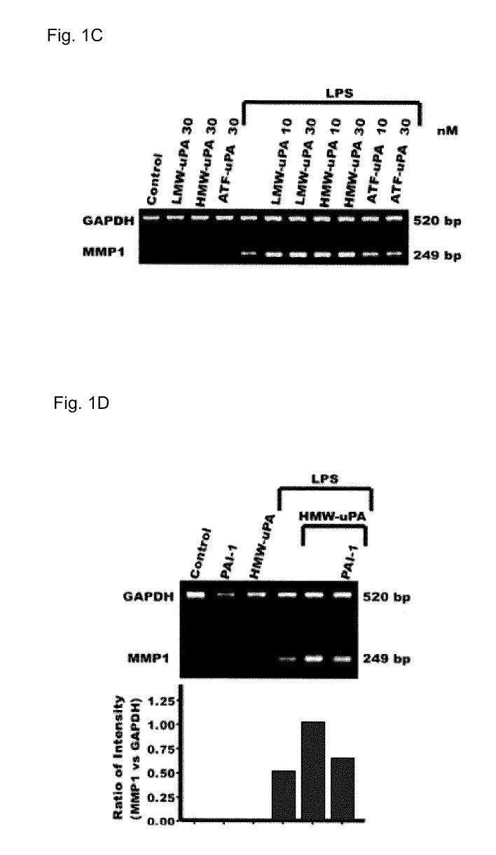

To determine the effect of uPA on human monocyte MMP-1 production, the catalytically active-two chain form of uPA (HMW-uPA) was added to control monocytes (not treated with LPS) or monocytes activated by LPS. HMW-uPA caused a dose-dependent increase in the protein levels of MMP-1 by LPS stimulated monocytes, whereas it did not induce MMP-1 in unstimulated monocytes (FIG. 1A). The majority of MMP-1 produced by monocytes is detected in the active 45 and 43 kDa form (ACL). In some studies, depending on the time of incubation and the donor, bands corresponding to procollagenase (PCL) were detected. The role of catalytic activity and/or binding to uPA receptor (uPAR) in the increased MMP-1 production was evaluated by comparing HMW-uPA, LMW-uPA and ATF-uPA. All studies are representative of three independent studies, an example of this is shown in the comparison of MMP-1 production by monocytes from three donors that were cultured in the presence or absence of LPS and the indicated concentrations of LMW-uPA, HMW-uPA and ATF-uPA (FIG. 1B). Catalytically active HMW-uPA, which binds to uPAR, and LMW-uPA, which does not bind to uPAR, increased LPS-induced production of MMP-1 protein. In contrast, ATF-uPA which lacks the catalytic domain, but binds to uPAR, did not stimulate MMP-1 production. The requirement of catalytically active uPA in the induction of MMP-1 expression by monocytes was also verified at the mRNA level (FIG. 1C) as well as in studies with a PAI-1 and HMW-uPA complex (FIG. 1D), which binds uPAR but is inactive. These findings demonstrate that the increased production of monocyte MMP-1, mediated by uPA, is dependent on the catalytic activity of this enzyme and does not require signaling through uPAR. These findings suggest that monocytes associated with inflammation or tumors may employ catalytically active uPA to increase MMP-1 production.

### Example 3

### Plasmin Stimulates MMP-1 Production

This example illustrates that uPA stimulation of MMP-1 production is mediated by plasmin.

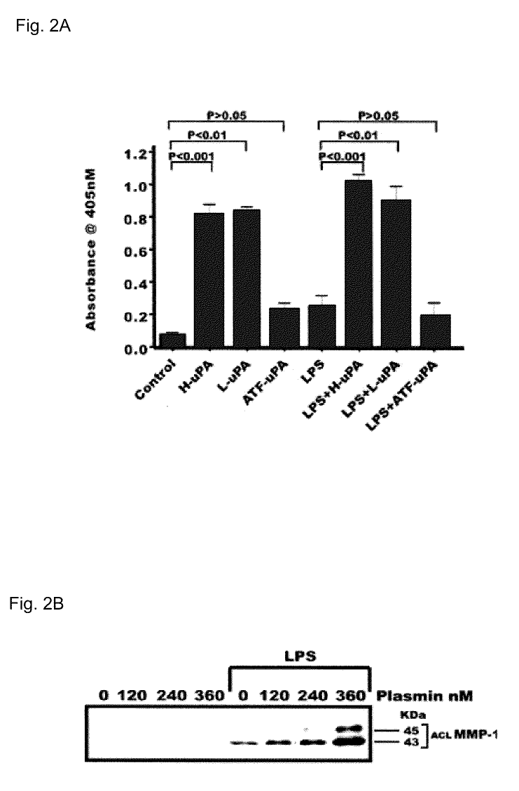

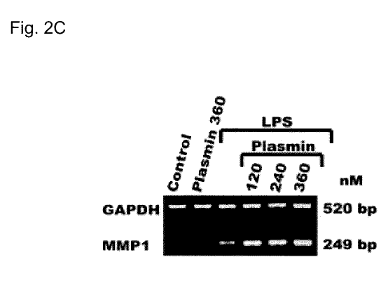

Stimulation of MMP-1 by catalytically active HMW-uPA and LMW-uPA but not ATF-uPA indicated that uPA was mediating its effect through the generation of plasmin from cell bound plasminogen. This was examined with a cell-based assay to determine plasmin levels in monocyte cultures. Addition of HMW-uPA or LMW-uPA to either control or LPS-stimulated monocytes induced a significant increase in plasmin activity (FIG. 2A). In contrast, ATF-uPA caused a slight, if any, increase in plasmin activity in unstimulated or LPS-stimulated monocytes.

Plasmin was added to monocyte cultures to determine its direct effect on MMP-1 production. Plasmin caused a dose-dependent increase in MMP-1 in LPS-stimulated monocytes, as shown at the protein and mRNA level, but did not induce MMP-1 in control (unstimulated) monocytes (FIGS. 2B and C). These findings demonstrate plasmin is generated from cell associated plasminogen by uPA and that plasmin requires a co-stimulant, such as LPS, to enhance MMP-1 production by monocytes. These findings suggest that plasmin may be capable of stimulating MMP-1 production in monocytes associated with inflammation or tumors.

### Example 4

### Plasmin Stimulates MMP-1 Production by Annexin A2

This example illustrates that plasmin induces MMP-1 production in monocytes through annexin A2.

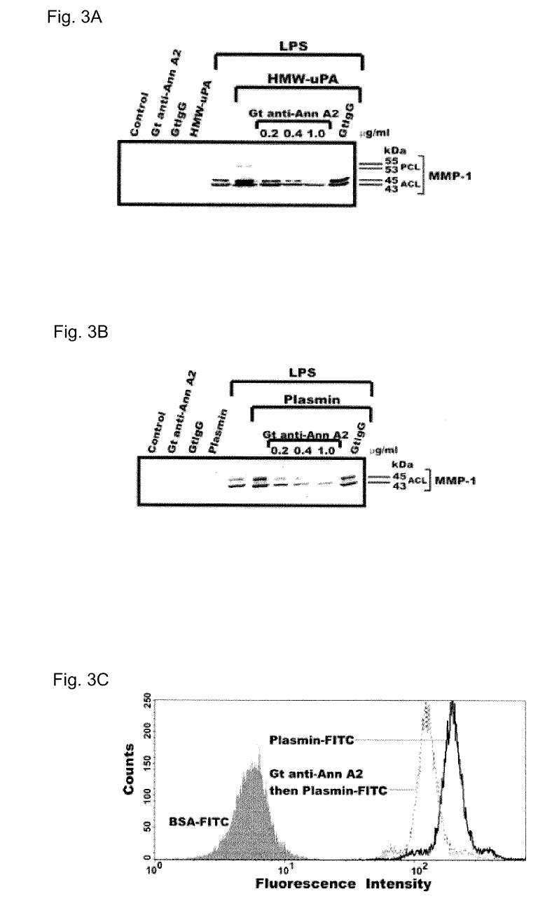

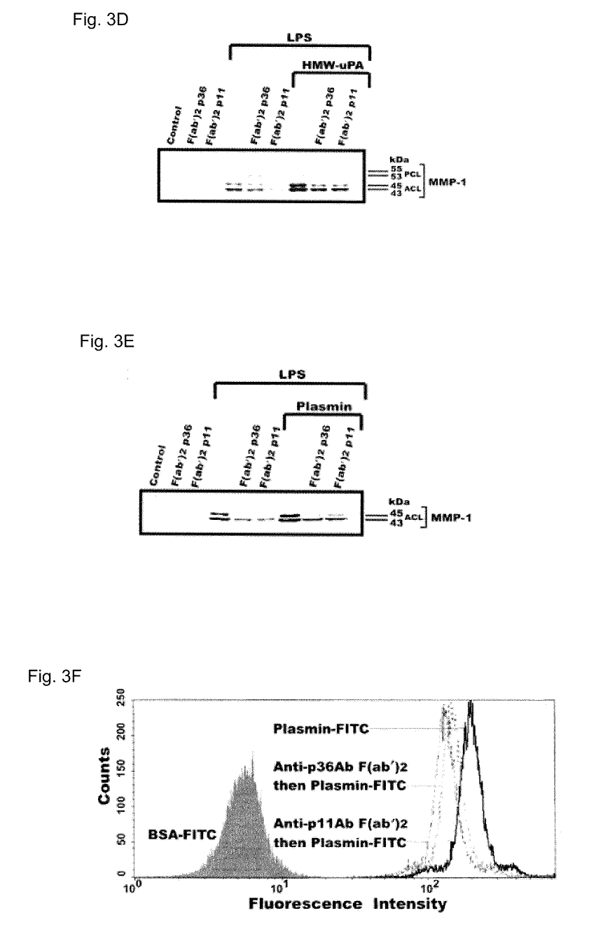

The heterotetramer of annexin A2 can serve as a receptor for plasmin on monocytes. To examine if plasmin stimulated MMP-1 production through annexin A2, a polyclonal antibody against annexin A2 was added to the monocyte cultures. This antibody caused a dose-dependent inhibition of HMW-uPA and plasmin-enhancement of MMP-1, whereas the isotype IgG had no effect (FIGS. 3A and B). Next F(ab′)₂ fragments against annexin A2 (p36) and S100A10 (p11), components of the annexin A2 heterotetramer, were used to determine if both components were involved in the induction of MMP-1. Both antibodies inhibited MMP-1 production induced by LPS, and the enhancement by HMW-uPA and plasmin (FIGS. 3D and E). Further evidence of the interaction of plasmin with annexin A2 was determined by flow cytometry analysis. Preincubation of monocytes with the polyclonal antibody against annexin A2 (FIG. 3C) or the monoclonal antibodies against p36 (annexin A2) or p11 (S100A10) (FIG. 3F) blocked the binding of fluorescein isothiocyanate (FITC)-labeled plasmin to monocytes. These findings demonstrate that uPA generation of endogenous plasmin or exogenous plasmin induction of MMP-1 production occurs through components that form the annexin A2 heterotetramer. These findings suggest that plasmin-mediated stimulation of MMP-1 production in monocytes associated with inflammation or tumors may occur through the annexin A2 heterotetramer.

### Example 5

### Role of PGE2 and MAPKs in Plasmin and HMW-uPA Signaling

This example illustrates that plasmin and HMW-uPA signaling leading to MMP-1 production occurs through prostaglandin-E2 (PGE₂) and MAPKs.

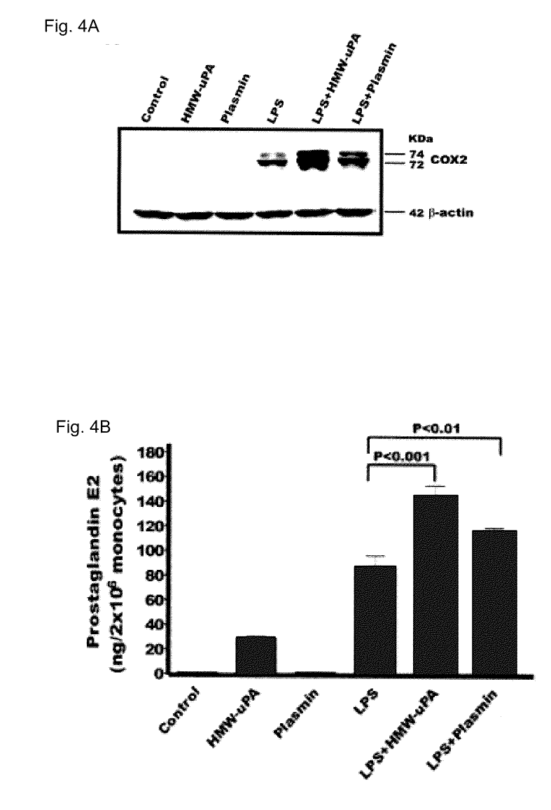

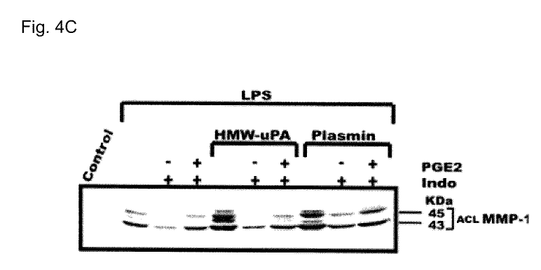

The production of MMP-1 by activated monocytes is regulated, in part, by PGE₂ which is synthesized as a result of the induction of cyclooxygenase-2 (COX-2) (Mertz et al., J. Biol. Chem. 269:21322-21329, 1994). Western blot analysis of the protein levels of COX-2 in the membranes of the monocytes revealed that the LPS-induced COX-2 was increased by HMW-uPA or plasmin (FIG. 4A), which corresponded to increased PGE₂ levels in the monocyte culture supernatants (FIG. 4B). PGE₂ was also detected in control monocytes treated with HMW-uPA, most likely derived from COX-1 since COX-2 was not induced. The LPS-stimulated increase in MMP-1 and the further enhancement of MMP-1 by HMW-uPA or plasmin were inhibited by indomethacin, which was restored, in part, by the addition of PGE₂ (FIG. 4C). These studies suggest that HMW-uPA or plasmin induction of PGE₂ is involved in the increase of MMP-1.

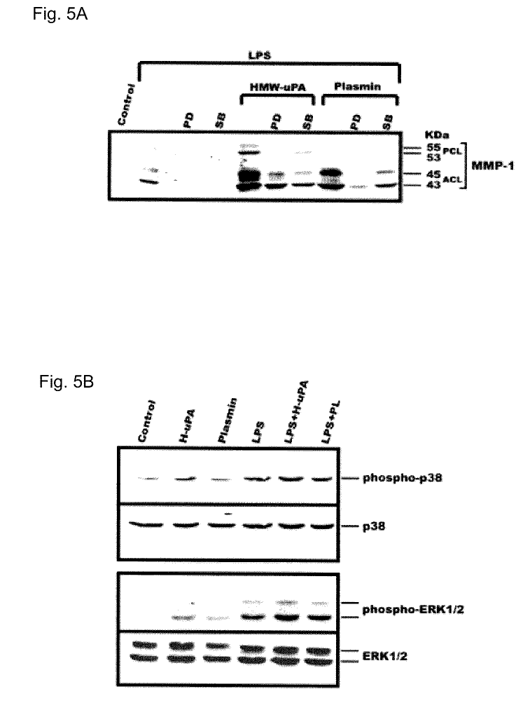

To determine if HMW-uPA generated plasmin or the direct addition of plasmin also induced MMP-1 through MAPKs, inhibitors of ERK1/2 (PD98059) (PD) or p38 (SB203580) (SB) were added to monocyte cultures. Both MAPK inhibitors suppressed the enhancement of MMP-1 by HMW-uPA or plasmin (FIG. 5A). HMW-uPA or plasmin also induced increases in the phosphorylation of p38 and ERK1/2 in LPS-stimulated monocytes (FIG. 5B). HMW-uPA also caused a slight increase in the phosphorylation of both MAPKs in unstimulated monocytes. These studies indicate that p38 and ERK1/2 were involved in mediating the induction of MMP-1 by plasmin. These findings suggest that plasmin-mediated stimulation of MMP-1 production in monocytes associated with inflammation or tumors may involve p38 and ERK1/2.

### Example 6

### Inactive Plasmin Inhibits MMP-1 Production

This example illustrates that inactive plasmin blocks plasmin-induced synthesis of MMP-1 by monocytes.

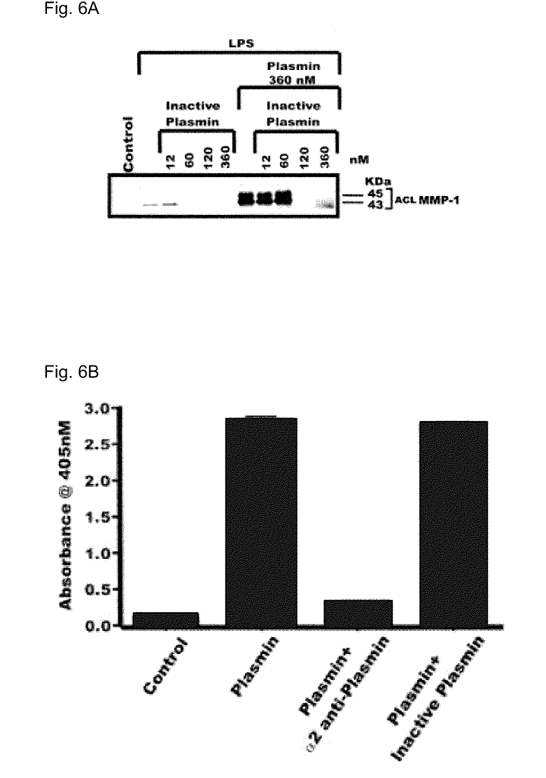

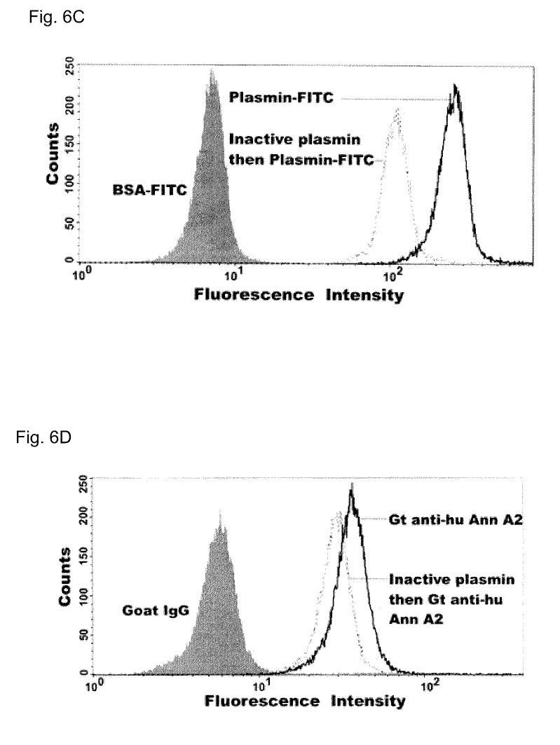

The requirement of catalytically active plasmin was required for the induction of MMP-1 synthesis was determined. Inactive plasmin, in which the catalytic site had been irreversibly blocked with a peptide inhibitor, was added to monocytes prior to the addition of LPS or LPS plus plasmin. Inactive plasmin inhibited the production of MMP-1 by LPS and LPS plus plasmin treated monocytes (FIG. 6A). This inhibition was not related to direct blocking of plasmin activity as shown in a cell free plasmin activity assay in which the combination of inactive plasmin and plasmin exhibited the same activity as plasmin alone (FIG. 6B). FACS analysis revealed that inactive plasmin blocked the binding of FITC-labeled plasmin (FIG. 6C) and the binding of antibodies against annexin A2 to monocytes (FIG. 6D). These findings demonstrate that inactive plasmin can function as an effective inhibitor of plasmin-mediated signaling in monocytes, including monocytes associated with inflammation or tumors. Thus, these studies indicate that an agent including inactive plasmin can be of use to suppress inflammation or a tumor associated with increased MMP-1 production.

### Example 7

### Treatment of Inflammation in a Human Subject

This example describes a method that can be used to treat inflammation in a human subject by administration of an agent that inhibits or suppresses the activation of annexin A2 receptor by plasmin. Although particular methods, dosages, and modes of administrations are provided, one skilled in the art will appreciate that variations can be made without substantially affecting the treatment.

Based upon the teaching disclosed herein, inflammation, such as inflammation associated with a disease (e.g., Exemplary inflammatory diseases affecting mammals include rheumatoid arthritis, osteoarthritis, inflammatory lung disease (including chronic obstructive pulmonary lung disease), inflammatory bowl disease (including ulcerative colitis and Crohn's Disease), periodontal disease, polymyalgia rheumatica, atherosclerosis, systemic lupus erythematosus, systemic sclerosis, Sjogren's Syndrome, asthma, allergic rhinitis, and skin disorders (including dermatomyositis or psoriasis) or a tumor, such as cancer of the lung, breast, ovaries, stomach, colon or esophagus), can be treated by administering a therapeutically effective amount of an agent including inactive plasmin that specifically inhibits the activation of the annexin A2 receptor (such as by interfering with the binding of such receptor with plasmin) thereby inhibiting or reducing MMP-1 production which in turn suppresses or eliminates the inflammation.

Briefly, the method can include screening subjects to determine if they are in need of inflammation suppression. Subjects having an inflammation or at risk of developing an inflammation are selected. In one example, subjects are diagnosed with the inflammatory condition by clinical signs, laboratory tests, or both. For example, periodontal disease can be diagnosed by clinical inspection of the gums for erythema, edema and recession of the gums from the teeth. Rheumatoid arthritis can be detected by characteristic clinical signs, such as red, swollen, painful and tender joints in a subject with an elevated sedimentation rate and/or positive rheumatoid factor and/or citrulline antibody. Alternatively, a subject known or at risk for inflammation or having increased levels of MMP-1 production in their blood (as detected with an enzyme-linked immunosorbent assay, Western blot, immunofluorescence assay, or nucleic acid testing) are selected.

In one example, a clinical trial includes half of the subjects following the established protocol for treatment of an inflammation (such as an anti-inflammatory therapy). The other half follows the established protocol for treatment of the inflammation (such as treatment with anti-inflammatory compounds) in combination with administration of the agents including inactive plasmin. In another example, a clinical trial includes half of the subjects following the established protocol for treatment of an inflammation (such as an anti-inflammatory therapy). The other half receives an agent including inactive plasmin.

### Screening Subjects

In particular examples, the subject is first screened to determine if they have an inflammation or are at risk of developing an inflammation. The inflammation may be musculoskeletal, neurological, cardiovascular, urological, gynecological, ophthalmic, dental, gastrointestinal, otological, dermatological, or respiratory. Inflammation may be determined or measured by many methods well known in the art, such as a clinical presentation that includes features such as pain and swelling, the number of white blood cells, the number of polymorphonuclear cells (PMN), a measure of the degree of PMN activation, such as luminal enhanced-chemiluminescence, a measure of the amount of cytokines present, detection of increased levels of MMP-1, or any combination thereof. For example, the detection of at least a 2-fold increase, such as a 5-fold, 10-fold increase in MMP-1 levels is indicative that the subject has an inflammation and is a candidate for receiving the therapeutic compositions disclosed herein.

In additional examples, screening of a subject further includes determining if the subject has an inflammatory disease or a disease associated with inflammation such as rheumatoid arthritis, osteoarthritis, inflammatory lung disease (including chronic obstructive pulmonary lung disease), inflammatory bowl disease (including ulcerative colitis and Crohn's Disease), periodontal disease, polymyalgia rheumatica, atherosclerosis, systemic lupus erythematosus, systemic sclerosis, Sjogren's Syndrome, asthma, allergic rhinitis, and skin disorders (including dermatomyositis and psoriasis). In a particular example, a subject that has an inflammatory disease or a disease associated with inflammation is a candidate for receiving one of the therapeutic agents disclosed herein.

In further examples, screening of a subject includes determining if the subject has a blood coagulation problem or requires thrombolysis or lysis of fibrin. In a certain example, a subject that is not afflicted with a blood coagulation problem or in need of thrombolytic therapy is a candidate for receiving one of the therapeutic agents provided herein.

Pre-screening is not required prior to administration of the therapeutic agents disclosed herein (such as those that including inactive plasmin).

### Pre-Treatment of Subjects

In particular examples, the subject is treated prior to administration of an agent that includes inactive plasmin. For example, the subject can be treated with an established protocol for treatment of an inflammatory disease (such as rheumatoid arthritis, cancer, atherosclerosis or a periodontal disease) prior to the administration of an agent including inactive plasmin. However, such pre-treatment is not always required, and can be determined by a skilled clinician.

### Administration of Therapeutic Agents

Following subject selection, a therapeutic effective dose of the agent including inactive plasmin is administered to the subject (such as a human either at risk for developing an inflammation or known to have an inflammation). For example, a therapeutic effective dose of an agent including inactive plasmin is administered to the subject to reduce or inhibit MMP-1 production (as described in detail above in the Section II., Methods of Suppressing Inflammation). Additional agents, such as anti-inflammatory agents, can also be administered to the subject simultaneously or prior to or following administration of the disclosed agents. Administration can be achieved by any method known in the art, such as oral administration, inhalation, intravenous, intramuscular, intraperitoneal, or subcutaneous (as described above in Administration Section).

The amount of the composition administered to prevent, suppress, inhibit, and/or treat inflammation or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., inflammation) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a daily, weekly, or monthly repeated administration protocol). In one example, a therapeutic agent that includes inactive plasmin is administered intravenously to a human. As such, these compositions may be formulated with an inert diluent or with a pharmaceutically acceptable carrier.

Administration of the therapeutic compositions can be taken long term (for example over a period of months or years).

### Assessment

Following the administration of one or more therapies, subjects having inflammation (for example, inflammation associated with a disease) can be monitored for reductions in MMP-1 levels, decreases in inflammation or in one or more clinical symptoms associated with the inflammation. In particular examples, subjects are analyzed one or more times, starting 7 days following treatment. Subjects can be monitored using any method known in the art. For example, biological samples from the subject, including blood, can be obtained and alterations in MMP-1 levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in inflammation.

### Studies in Animal Models

One of skill in the art will appreciate that the disclosed agents including inactive plasmin can be tested for safety in animals, and then used for clinical trials in animals or humans. In one example, mouse models of inflammation are employed to determine therapeutic value of the disclosed agents. For example, arthritis will be induced in mice by immunization with type II collagen followed by subsequent injection of type II collagen 21 days later. (Kubota et al., Arthritis Res. Ther. 9 (5): R97, 2007).

### Particular Regimen

A particular example of treatment with a plasmin inhibitor is to select a subject with rheumatoid arthritis and administer an agent including inactive plasmin by slow infusion at a dose of 1 to 2 mg/kg for 30 minutes and then assessing the inflammation 7 days following treatment. The inactive plasmin is prepared from Lys-plasmin by active site-specific inactivation with a Phe-Phe-Arg chloromethyl ketone or by a point mutation in the catalytic site of plasmin. In one example, the effectiveness of the treatment is determined by measuring rheumatoid factor antibodies in a blood sample taken from the subject at the 7-day time point. An RF value less than that detected in the 95th percentile is considered to be an effective treatment. In other examples, the effectiveness of the treatment is determined by measuring anti-citrullinated protein antibodies (ACPA) by use of an anti-CCP (cyclic citrullinated peptide) test prior to and following treatment. A two-fold reduction in ACPA antibodies is considered to be an effective treatment.

In another particular example, treatment with a plasmin inhibitor is to select a subject with a musculoskeletal disorder, such as systemic lupus erythematosus and administer an agent including inactive plasmin by slow infusion at a dose of 1 to 2 mg/kg for 30 minutes and then assessing the inflammation 7 days following treatment. The inactive plasmin is prepared from Lys-plasmin by active site-specific inactivation with a Phe-Phe-Arg chloromethyl ketone. In one example, the effectiveness of the treatment is determined by measuring antinuclear antibodies (ANA) in a blood sample taken from the subject prior to treatment and at the 7-day time point.

### Example 8

### Treatment of a Tumor in a Human Subject

This example describes a method that can be used to treat a primary or metastatic tumor in humans by administration of an agent including inactive plasmin. Although particular methods, dosages, and modes of administrations are provided, one skilled in the art will appreciate that variations can be made without substantially affecting the treatment.

In an example, human subjects are treated intravenously with a therapeutically effective dose of an agent including inactive plasmin, for example for a period of at least 6 months, at least one year, at least 2 years, or at least five years. Administration of the agent including inactive plasmin can be used in conjunction with normal cancer therapy (for example rather than replacing the therapy). Thus, the therapeutic agent can be added to the usual and customary chemotherapy, surgery and/or radiation treatments conventionally used for the particular tumor type, such as cancer. Administration of the therapeutic agent can be continued after chemotherapy and radiation therapy was stopped and can be taken long term (for example over a period of months or years).

Briefly, the method can include screening subjects to determine if they have a tumor, such as primary or metastatic tumor. Subjects having a tumor are selected. In one example, subjects who have a tumor and increased levels of MMP-1 in their serum are selected. In a clinical trial, half of the subjects follow the established protocol for treatment of a tumor, such as cancer (including a normal chemotherapy/radiotherapy/surgery regimen). The other half follow the established protocol for treatment of the tumor (such as a normal chemotherapy/radiotherapy/surgery regimen) in combination with administration of the therapeutic agent including inactive plasmin described above. In some examples, the tumor is surgically excised (in whole or part) prior to treatment with the therapeutic agent.

### Screening Subjects

In particular examples, the subject is first screened to determine if they have a tumor, such as cancer (e.g., cancer of the lung, breast, ovaries, stomach, colon or esophagus). Examples of methods that can be used to screen for cancer include a combination of ultrasound, tissue biopsy and examination of MMP-1 levels. Increased levels of MMP-1 (such as at least a two-fold increase as compared to MMP-1 levels in non-tumor cells) and a positive imaging result indicate that the subject has a tumor that can be treated with the disclosed therapies.

However, such pre-screening is not required prior to administration of an agent including inactive plasmin.

### Pre-Treatment of Subjects

In particular examples, the subject is treated prior to administration of a therapeutic agent that includes inactive plasmin. For example, the tumor can be surgically excised (in total or in part) prior to administration of the agent including inactive plasmin. In addition, the subject can be treated with an established protocol for treatment of the particular tumor present (such as a normal chemotherapy/radiotherapy regimen). However, such pre-treatment is not always required, and can be determined by a skilled clinician.

### Administration of Therapeutic Agents

Administration can be achieved by any method known in the art, such as oral administration, inhalation, or inoculation (such as described in detail above). In one example, the therapeutic agent includes inactive plasmin. The amount of inactive plasmin administered is sufficient to treat a subject having tumor, such as cancer. An effective amount can being readily determined by one skilled in the art, for example using routine trials establishing dose response curves. In addition, particular exemplary dosages are provided above (see Administration Section). The therapeutic agents can be administered in a single dose delivery, via continuous delivery over an extended time period, in a repeated administration protocol (for example, by a, daily, weekly, or monthly repeated administration protocol). In one example, a therapeutic agent that includes inactive plasmin is administered intravenously to a human. As such, this agent may be formulated with an inert diluent or with a pharmaceutically acceptable carrier.

### Assessment

Following the administration of one or more therapies, subjects having a tumor (for example, cancer) can be monitored for tumor treatment, such as regression or reduction in metastatic lesions. In particular examples, subjects are analyzed one or more times, starting 7 days following treatment

Subjects can be monitored using any method known in the art. For example, diagnostic imaging can be used (such as x-rays, CT scans, MRIs, ultrasound, fiberoptic examination, and laparoscopic examination), as well as analysis of biological samples from the subject (for example, analysis of blood, tissue biopsy, or other biological samples), such as analysis of the type of cells present, or analysis for a particular tumor marker. In one example, if the subject has a metastatic cancer, assessment can be made using ultrasound, MRI, or CAT scans, and analysis of the type of cells contained in a tissue biopsy.

### Evaluation Following Treatment

During or following therapeutic treatment, subjects can be monitored for the response of their tumor(s) to the therapy.

Subjects can receive a complete physical evaluation, complete blood count, acute care, and appropriate evaluations of all evaluable lesions (for example by x-ray, MRI, CT scan, ultrasound) are obtained every 6-12 weeks during the first six months of therapy and if stable, every 3-6 months thereafter. Other evaluations can be performed as indicated by symptoms or physical findings.

### Additional Treatment

In particular examples, if subjects are stable or have a minor, mixed or partial response to treatment, they can be re-treated after re-evaluation with the same schedule and preparation of agent that they previously received for up to a year of total therapy.

A mixed response is the shrinkage of some lesions but an increase in others. Subjects with mixed responses may only receive treatment for an additional 2-3 months without showing true disease stability or a bona fide minor or major response (e.g., no further progression). Two re-treatment cycles can be given following a complete response.

### Studies in Animal Models

One of skill in the art will appreciate that the disclosed agents including inactive plasmin can be tested for safety in animals, and then used for clinical trials in animals or humans. In one example, genetically engineered mouse models of cancer are employed to determine therapeutic value of the disclosed agents. For example, the conditional expression of K-ras in an epithelial compartment in mice that includes stems cells is sufficient to rapid development of squamous cell carcinogenesis (Vitale-Cross et al., Cancer Res. 64(24): 8804-8807, 2004).

### Particular Regimen

A particular example of treatment with a plasmin inhibitor is to select a subject with a metastatic tumor and administer an agent including inactive plasmin by slow infusion with an initial loading dose of 4 mg/kg over a period of 90 minutes, followed by weekly maintenance doses for 4-8 weeks of 2 mg/kg infused over a 30 minute period. Metastatic lesions are evaluated weekly by diagnostic imaging techniques including ultrasound, MRI, or CAT scans. The inactive plasmin is prepared from Lys-plasmin by active site-specific inactivation with a Phe-Phe-Arg chloromethyl ketone. A reduction in the metastatic lesions by at least 2-fold is an effective therapy.

### Example 9

### Method of Modulating Annexin A2 Receptor Activity

This example illustrates the methods of modulating annexin A2 receptor activity via administering an agent including inactive plasmin.

Based upon the teachings disclosed herein, annexin A2 receptor activity can be modulated, such as reduced or inhibited, by contacting a cell with an effective amount of an agent including inactive plasmin in which the agent specifically inhibits the activation of the annexin A2 receptor, thereby inhibiting MMP-1 production (as described in detail in Section IV., Example 1 and Example 6). The cell can also be contacted with an effective amount of an additional agent, such as anti-inflammatory or anti-neoplastic agent. The cell can be in vivo or in vitro. In particular examples, the method includes modulating, such as inhibiting or decreasing, annexin A2 receptor activity associated with inflammation or a tumor. For example, a cell, such as a tumor cell, cancer cell, or white blood cell, is contacted with a therapeutically effective dose of an agent including inactive plasmin. The inactive plasmin can modulate the activity of the annexin A2 receptor (e.g., inhibiting or reducing the ability of plasmin to bind to such receptor), thereby inhibiting or reducing MMP-1 production. This method can be used to suppress inflammation associated with a pathological condition, such as atherosclerosis, periodontal disease, rheumatoid arthritis, or a tumor.

### Example 10

### Screening for Anti-Inflammatory Agents

This example describes methods that can be used to identify agents to treat inflammation.

According to the teachings herein, one or more agents for treating inflammation can be identified by determining whether an agent irreversibly inhibits plasmin activity. For example, whether an agent irreversibly inhibits the ability of plasmin to bind to an annexin A2 receptor can be determined. The method can include selecting an agent that irreversibly inhibits plasmin activity. The method can also include contacting a cell, such as a cell expressing at an annexin A2 receptor, with the selected agent under conditions sufficient for the agent to alter the activity of plasmin. The method can also include detecting a decrease in the binding of a plasmin to annexin A2 receptor relative to a control. A decrease in the binding of plasmin to annexin A2 relative to a control identifies the agent as one that is useful to treat inflammation. Decreased binding can be detected by an in vitro assay in which the activity of plasmin in the presence and absence of the one or more test agents can be determined. Various types of in vitro assays may be employed to identify agents to treat inflammation including, but not limited to, binding assays, standard Western blot or immunoassay techniques and other well known assays to those of skill in the art. However, the disclosure is not limited to particular methods of detection.

In a specific example, a library of plasmin inhibitors are screened for their effect on plasmin activation of the annexin A2 receptor. Regardless of the assay technique, agents that cause at least a 2-fold decrease, such as at least a 3-fold decrease, at least a 4-fold decrease, or at least a 5-fold decrease in the activity, such as binding of plasmin to annexin A2 receptor, are selected for further evaluation.

Candidate agents also can be tested in additional cell lines and animal models of inflammation to determine their therapeutic value. The agents also can be tested for safety in animals, and then used for clinical trials in animals or humans. In one example, mouse models of inflammation are employed to determine therapeutic value of test agents. For example, arthritis will be induced in mice by immunization with type II collagen followed by subsequent injection of type II collagen 21 days later. (Kubota et al., Arthritis Res. Ther. 9 (5): R97, 2007).

The disclosure described and claimed herein is not to be limited in scope by the specific embodiments herein disclosed, since these embodiments are intended as illustrations of several aspects of the disclosure. Any equivalent embodiments are intended to be within the scope of this disclosure. Indeed, various modifications of the disclosure in addition to those shown and described herein will become apparent to those skilled in the art from the foregoing description. Such modifications are also intended to fall within the scope of the appended claims. In the case of conflict, the present disclosure including definitions will control.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

We claim:

1. A method of suppressing inflammation in a subject, comprising:
selecting the subject in need of suppression of inflammation; and
inhibiting plasmin activity in the subject to decrease matrix metalloproteinase production,
thereby suppressing the inflammation.

2. The method of claim 1, wherein inhibiting plasmin activity in the subject comprises administering to the subject an agent comprising irreversible inactive plasmin in a therapeutically effective amount to decrease matrix metalloproteinase production.

3. The method of claim 2, wherein the agent interacts with an annexin A2 receptor inhibiting the ability of plasmin to bind to the annexin A2 receptor.

4. The method of claim 1, wherein the matrix metalloproteinase is matrix metalloproteinase-1.

5. The method of claim 1, wherein the inflammation is associated with a disease.

6. The method of claim 5, wherein the disease is at least one of atherosclerosis, a periodontal disease, rheumatoid arthritis or a tumor.

7. The method of claim 6, wherein the tumor is cancer.

8. The method of claim 1, wherein selecting the subject in need of suppression of inflammation comprises selecting a subject not in need of clot lysis.

9. The method of claim 1, wherein selecting the subject in need of suppression of inflammation comprises selecting a subject having a primary inflammatory disorder.

10. A method of suppressing a tumor in a subject, comprising:
selecting the subject in need of suppression of the tumor; and
administering an agent comprising inactive plasmin at a therapeutically effective concentration to a subject to decrease matrix metalloproteinase production,
thereby suppressing the tumor.

11. The method of claim 10, wherein the matrix metalloproteinase is matrix metalloproteinase-1.

12. The method of claim 10, wherein the agent interacts with an annexin A2 receptor inhibiting the ability of plasmin to bind to the annexin A2 receptor and facilitate tumor cell invasion.

13. The method of claim 10, wherein the agent interacts with an annexin A2 receptor inhibiting the ability of plasmin to bind to the annexin A2 receptor and facilitate tumor cell metastasis.

14. The method of claim 10, wherein the tumor is cancer.

15. The method of claim 10, wherein the method further comprises administering one or more additional therapeutic agents at a therapeutically effective amount to the subject.

16. The method of claim 15, wherein the one or more additional therapeutic agents comprise one or more anti-neoplastic agents.

17. The method of claim 10, wherein the subject is a mammalian subject.

18. The method of claim 17, wherein the mammalian subject is a human subject.

19. A method for modulating annexin A2 receptor activity, comprising:
contacting a cell with a therapeutically effective concentration of an agent comprising inactive plasmin, wherein the inactive plasmin modulates the activity of an annexin A2 receptor and effects a change in the level of matrix metalloproteinase production by the treated cell relative to matrix metalloproteinase production in an untreated cell.

20. The method of claim 19, wherein the level of matrix metalloproteinase is the level of matrix metalloproteinase-1.

21. The method of claim 19, wherein the level of matrix metalloproteinase-1 production is decreased relative to an untreated cell.

22. The method of claim 19, wherein the cell is a tumor cell.

23. The method of claim 22, wherein the tumor cell is a cancer cell.

24. The method of claim 19, wherein the cell is a white blood cell.

25. A method of screening for an anti-inflammatory agent, comprising:
determining whether an agent irreversibly inhibits plasmin activity; and
selecting an agent that irreversibly inhibits plasmin activity.
